(19) United States
(12) Patent Application Publication
(10) Pub. No.: US 2023/0287167 A1
(43) Pub. Date: Sep. 14, 2023

(54) SYNTHESIS OF HIGH MOLECULAR WEIGHT AND STRENGTH POLYISOBUTYLENE-BASED POLYURETHANES AND USE THEREOF

(71) Applicants: THE UNIVERSITY OF AKRON, AKRON, OH (US); Cambridge Enterprise Limited, Cambridge,Cambridgeshire (GB)

(72) Inventors: Joseph KENNEDY, Akron, OH (US); Turgut NUGAY, Sariyer-Istanbul (TR); Nihan NUGAY, Sariyer-Istanbul (TR); Tejal DEODHAR, Akron, OH (US); Geoff MOGGRIDGE, Cambridge (GB); Ruhi PATEL, Cambridge (GB)

(21) Appl. No.: 18/040,465

(22) PCT Filed: Aug. 5, 2021
(86) PCT No.: PCT/US2021/044616

§ 371 Date: Feb. 3, 2023

Related U.S. Application Data
(60) Provisional application No. 63/061,852, filed on Aug. 6, 2020.

(51) Int. Cl. C08G 18/64; C08G 18/76; A61L 27/18

(52) U.S. Cl. CPC C08G 18/64; C08G 18/7692; A61L 27/18; A61L 2430/20

(57) ABSTRACT

A method of preparing a polyisobutylene-based polyurethane for making heart valves is disclosed, wherein the method includes providing a polyisobutylene (PIB) polymer, freshly distilling a diisocyanate compound to create a freshly distilled diisocyanate and providing a chain extender. When the polyisobutylene polymer, the freshly distilled diisocyanate, and the chain extender are combined together by mixing, the created polyisobutylene-based polyurethane exhibits a higher number average molecular weight, a higher ultimate strength, a higher elongation, and a greater toughness than a polyisobutylene-based polyurethane made without a freshly distilled diisocyanate, which makes the polymer particularly useful as a bioprosthetic heart valve.

### FIELD OF THE INVENTION

The present invention generally relates to novel polyisobutylene-based polyurethanes and the method of making the same. More particularly, it has been found that under synthesis conditions previously recognized as unattainable and unrealizable, the polyisobutylene-based polyurethanes of the present invention have a higher number average molecular weight, a higher ultimate strength, a higher elongation, and a greater toughness than previous polyisobutylene-based polyurethanes known to exist. Specifically, it has been found that the use of a freshly distilled diisocyanate and an increased solid content of the synthesis solution to the limit beyond which increased viscosity prevents stirring provides the polyisobutylene-based polyurethanes of the present invention with their enhanced characteristics. Such polyurethanes are particularly useful as heart valves.

### BACKGROUND OF THE INVENTION

Over 500,000 prosthetic heart valves are implanted per year. Clinically available prosthetic valves are lifesaving, but imperfect. Mechanical valves require lifelong anti-coagulation medication to be taken by the users, while also carrying associated bleeding and thrombosis risks. Bioprosthetic valves show structural deterioration, so they ultimately require another operation to be undertaken. Polymeric heart valves have the potential to achieve longer durability than their counterparts, without also needing the user to take anti-coagulation medicine. For this reason, there has been interest since the 1970's in producing a viable polymeric valve; but none has achieved regulatory approval for clinical application.

With improvements in materials, manufacturing techniques, and modeling, there has been a resurgence of research into polymeric valves, with several promising prototypes emerging. Once such prototype is the Foldax® Tria™ polyurethane valve, which started human trials in July 2019. This is not a polyisobutylene-based polyurethane valve. Much attention has focused on thermoplastic polyurethanes because of their good Physio-chemical properties and their ease of processing. Recent formulations, such as Foldax® Tria™ polyurethanes have shown improved biocompatibility and stability. Nonetheless, to date, polyurethanes have suffered from calcification and/or gradual oxidation/degradation in vivo, resulting in mechanical failures and/or thrombosis.

Polyisobutylene-based polyurethanes (PIB-PUs) containing 70% polyisobutylene are known to be bioinert, meaning they are both biocompatible and biostable, and calcification resistance thermoplastic elastomers usable in long-term implantable medical devices. These 70% polyisobutylene PIB-PUs are also able to be made at a modest cost. These thermoplastic elastomers also exhibit an exceptional combination of hydrolytic, oxidative, and enzymatic resistance while at the same time having exceptional softness, barrier properties, low creep, and low cell adhesion. Furthermore, the inert continuous soft segments made from —CH₂—C(CH₃)₂, shield the environmentally vulnerable hard segments made from urethanes (—NHCOO—) while also leading to desirable combinations of properties.

However, they do come with a downside. Namely, present day inert PIB soft segments lack hydrogen bond accepting sites. Therefore, the strength of these PIB-PUs has heretofore been inherently lower than those of conventional polyurethanes whose soft segments contain nucleophilic sites (i.e., oxygen or nitrogen sites). Therefore, there is a need in the art to increase the strength of 70% polyisobutylene PIB-PUs, while maintaining the biocompatibility and biostability, as well as the calcification resistance and excellent hydrolytic, oxidative, and enzymatic resistance while at the same time having exceptional softness, barrier properties, low creep, and low cell adhesion.

### SUMMARY OF THE INVENTION

Advantageously, it has been found that the use of freshly distilled diisocyanate in reaction with polyisobutylene diols and, optionally, a chain extender, provides PIB-PUs that exhibit higher number average molecular weight, a higher ultimate strength, a higher elongation, and a greater toughness than a conventional PIB-PUs, i.e., those made without a freshly distilled diisocyanate. It will be appreciated that, by the term “freshly distilled,” it is meant that the diisocyanate, particularly, MDI, is distilled and then used or otherwise reacted with polyisobutylene diol within 1 or 2 hours after distillation to create polyisobutylene-based polyurethane. In other words, unlike all previous methods, the diisocyanate is not stored prior to being used in any manner known in the art, and is instead used within 2 hours, more preferably, within 1 hour, and even more preferably, within 30 minutes after distillation.

In addition, it has also been advantageously found that providing the polyisobutylene polymer is a solution of tetrahydrofuran (THF), wherein the concentration of the polyisobutylene polymer is, in one embodiment, at least 21.4 wt. % in THF, and in a second embodiment, greater than 21.4 wt. % in THF, and in a third embodiment, is at least 28 wt. % in THF, aids further in providing PIB-PUs that exhibit higher number average molecular weight, a higher ultimate strength, a higher elongation, and a greater toughness than a conventional PIB-PUs, i.e., those that do not include as high a concentration of PIB in THF as set forth above. It is noted that, with such high concentration of PIB polymer, it is nearly impossible to stir the PIB polymer after only 20 minutes upon reaction with the diisocyanate and/or chain extender. In contrast to other methods, this method allows the reaction to continue without stirring and adding minor amounts of THF so that the stirring may resume for about 3 additional hours.

In light of these advantages, an embodiment of the present invention provides a method of preparing a polyisobutylene-based polyurethane. The method includes providing a polyisobutylene (PIB) polymer, freshly distilling a diisocyanate compound to create a freshly distilled diisocyanate and providing a chain extender. When the polyisobutylene polymer, the freshly distilled diisocyanate, and the chain extender are combined together by mixing, the created polyisobutylene-based polyurethane exhibits a higher number average molecular weight, a higher ultimate strength, a higher elongation, and a greater toughness than a polyisobutylene-based polyurethane made without a freshly distilled diisocyanate.

Another embodiment of the present invention provides a method of preparing a polyisobutylene-based polyurethane as in any embodiment above, wherein the method further comprises the steps of providing a catalyst and combining said catalyst with the polyisobutylene polymer, the freshly distilled diisocyanate, and the chain extender.

Another embodiment of the present invention provides a method of preparing a polyisobutylene-based polyurethane as in any embodiment above, wherein the step of providing a catalyst includes selecting the catalyst from the group consisting of dibutyltin dilaurate (DBTDL), stannous octoate, bismuth/zinc, zirconium and bismuth organics including bismuth neodecanoate, zinc neodecanoate, zinc carboxylate, and bismuth carboxylate, vanadium organics, and cobalt organics.

Another embodiment of the present invention provides a method of preparing a polyisobutylene-based polyurethane as in any embodiment above, wherein the step of providing PIB polymer includes selecting a PIB-diol as the PIB polymer.

Another embodiment of the present invention provides a method of preparing a polyisobutylene-based polyurethane as in any embodiment above, wherein the step of freshly distilling a diisocyanate includes selecting methylene diphenyl diisocyanate to be the freshly distilled diisocyanate.

Another embodiment of the present invention provides a method of preparing a polyisobutylene-based polyurethane as in any embodiment above, wherein the step of providing a chain extender includes selecting butane diol as the chain extender.

Another embodiment of the present invention provides a method of preparing a polyisobutylene-based polyurethane as in any embodiment above, wherein the step of combining produces a polyisobutylene-based polyurethane having a number average molecular weight of greater than 100,000 Da.

Another embodiment of the present invention provides a method of preparing a polyisobutylene-based polyurethane as in any embodiment above, wherein the step of combining produces a polyisobutylene-based polyurethane having an ultimate strength of greater than 30 MPa.

Another embodiment of the present invention provides a method of preparing a polyisobutylene-based polyurethane as in any embodiment above, wherein the step of combining produces a polyisobutylene-based polyurethane having an elongation of greater than 600%.

Another embodiment of the present invention provides a method of preparing a polyisobutylene-based polyurethane as in any embodiment above, wherein the step of combining produces a polyisobutylene-based polyurethane having a toughness of greater than 4.00 J.

Another embodiment of the present invention provides a method of preparing a polyisobutylene-based polyurethane as in any embodiment above, wherein the step of providing PIB polymer includes providing said PIB polymer in a solution of THF and wherein the concentration of said PIB polymer is at least 21.4 wt. % in THF.

Another embodiment of the present invention provides a method of preparing a polyisobutylene-based polyurethane as in the embodiment above, wherein the step of providing PIB polymer includes providing said PIB polymer in a solution of THF at a concentration of greater than 21.4 wt. % in THF such that mixing during the combining step becomes impossible.

An embodiment of the present invention provides a polyisobutylene-based polyurethane. The polyisobutylene-based polyurethane comprises the reaction product of a polyisobutylene (PIB) polymer, a freshly distilled diisocyanate and a chain extender. The polyisobutylene-based polyurethane produces exhibits a higher number average molecular weight, a higher ultimate strength, a higher elongation, and a greater toughness than a polyisobutylene-based polyurethane made without a freshly distilled diisocyanate.

Another embodiment of the present invention provides a polyisobutylene-based polyurethane as in any embodiment above, wherein the reaction product further includes a catalyst selected from the group consisting of Dibutyltin dilaurate (DBTDL), stannous octoate, bismuth/zinc, zirconium and bismuth organics including bismuth neodecanoate, zinc neodecanoate, zinc carboxylate, and bismuth carboxylate, vanadium organics, and cobalt organics.

Another embodiment of the present invention provides a polyisobutylene-based polyurethane as in any embodiment above, wherein the polyisobutylene polymer is a PIB-diol.

Another embodiment of the present invention provides a polyisobutylene-based polyurethane as in any embodiment above, wherein the freshly distilled diisocyanate compound is methylene diphenyl diisocyanate.

Another embodiment of the present invention provides a polyisobutylene-based polyurethane as in any embodiment above, wherein the chain extender is butane diol.

Another embodiment of the present invention provides a polyisobutylene-based polyurethane as in any embodiment above, wherein the polyisobutylene-based polyurethane has a number average molecular weight of greater than 100,000 Da, an ultimate strength of greater than 30 MPa, an elongation of greater than 600%, and a toughness of greater than 4.00 J.

### BRIEF DESCRIPTION OF THE DRAWINGS

The FIGURE shows stress vs. strain traces of a PIB-PU obtained from this work, together with the highest quality PIB-PU reported to date, and a commercial silicon rubber-based polyurethane (Elast-Eon™)

### DETAILED DESCRIPTION OF ILLUSTRATIVE EMBODIMENTS

The present invention teaches a polyisobutylene-based polyurethane (PIB-PU) having at least a 70% polyisobutylene soft segment having a number average molecular weight (Mn) of greater than 100,000 Da, an ultimate strength of 32 MPa, and an elongation of 630%. The PIB-PU of the present invention, through its 70% PIB soft segment, is also bioinert and calcification resistant. The key parameters surrounding the production of a PIB-PU having such important characteristics are the precise stoichiometry of the polyurethane forming reaction, specifically the use of highly purified diisocyanate (methylene diphenyl diisocyanate, known as MDI), and the increased solid content of the synthesis solution to a limit beyond which increased viscosity prevents stirring.

The shape of the stress-strain trace of the formed PIB-PU of the present invention indicates a two-step failure starting with a reversible elastic (Hookian) region up to about a 50% yield, followed by a slower linearly increasing high modulus deformation region. This stress-strain trace suggests the strengthening of the PIB soft segments by entanglement/catenation and strengthening of the hard segments by progressively ordering of the urethane domains.

The maximum molecular weight ever reported for a PIB-PU containing a 70 wt. % PIB soft segment was about 70 kDa, the maximum tensile strength ever reported was about 26 MPa, and the maximum elongation ever reported was about 500%. By optimizing synthesis conditions, specifically using freshly distilled MDI, and increasing the PIB diol concentration in the synthesis solution, the PIB-PU's of the present invention have molecular weights of greater than 100 kDa, tensile strengths of about 32 MPa, elongation of about 630%, and a toughness of greater than 4.0 J.

Such PIB-PUs of the present invention are believed to be particularly useful as the polymer used as at least the flap(s) in a bioprosthetic heart valve. The heart valve of the present invention is believed to have longer durability than their counterparts, without also needing the user to take anti-coagulation medicine. The improvements in the materials and manufacturing processes of the present invention is believed to provide a polyisobutylene-based polyurethane valve having excellent mechanical properties and continued ease of processing. The PIB-based polyurethane of the present invention is believed to have excellent biocompatibility and stability for the heart valve, and does not suffered from calcification and/or gradual oxidation/degradation in vivo, which could result in mechanical failures and/or thrombosis.

A representative experiment used to produce PIB-PU's containing a 70 wt. % PIB soft segment will now be described. A flame dried glass vial equipped with a mechanical stirrer was charged with well dried PIB-diol (0.5 mmol, 1.5 g), freshly distilled MDI (2.025 mmol), and 4 mL distilled THF under a blanket of N₂. The system was stirred and heated to 65° C., then a catalyst solution (0.24 mL of a 25 mg Dibutyltin dilaurate (DBTDL)/5 mL THF) was added and stirred for one hour. A butane diol chain extender (1,525 mmol, 137.4 mg dissolved in 3 mL THF) was then added, and the system was further stirred for an additional 3 hours at 65° C. The system was then further diluted with additional THF and was then poured into a glass mold while still warm. The product was then slowly dried at room temperature for about 24 hours, and then the product was further dried in a vacuum for 2 days at 75° C.

The representative experiment discussed above discusses the use of “freshly distilled” MDI. Freshly distilled within the context of this application means that the MDI was distilled and then used within 1 to 2 hours after distillation to create the PIB-PU's of the present invention. Freshly distilled further defines that the freshly distilled MDI was not stored prior to being used to create the PIB-PU's of the present invention.

Although the above experiment discusses the use of DBTDL as the catalyst, in other embodiments of the present invention, other catalysts could be used such as stannous octoate, bismuth/zinc, zirconium and bismuth organics including bismuth neodecanoate, zinc neodecanoate, zinc carboxylate, and bismuth carboxylate, vanadium organics, and cobalt organics.

As stated above, it was determined that the purity of the MDI utilized to create the PIB-PU's of the present invention was vital to the advanced mechanical properties of the produced. Therefore, the effects of MDI pretreatment and the shelf life of the MDI prior to use was studied. The results of those studies can be appreciated by review of Table 1 below.

TABLE 1

| Experiment | | | | | |
|---|---|---|---|---|---|
| No. | 1 | 2 | 3 | 4 | 5 |
| MDI* Pre- | As | Distilled | Distilled | Freshly | Freshly |
| treatment | received. | then | then | distilled. | distilled. |
| | | stored. | stored. | | |
| MDI | N/A | 6 weeks | 3 weeks | N/A | N/A |
| Storage | | | | | |
| Time | | | | | |
| MDI | N/A | −12 | −12 | N/A | N/A |
| Storage | | | | | |
| Temp (° C.) | | | | | |
| PIB-Diol | 21.4 | 21.4 | 21.4 | 21.4 | 28.5 |
| Concentration | | | | | |
| in THF | | | | | |
| (wt. %) | | | | | |
| MW/MWD | 21/1.8 | 24/1.8 | 29/1.8 | 56/1.9 | 101/3.1 |
| (kDa/—) | | | | | |
| Stress at | 9 | 12 | 14 | 22 | 32 |
| Break | | | | | |
| (MPa) | | | | | |
| Strain at | 110 | 141 | 190 | 480 | 630 |
| Break (%) | | | | | |
| Energy to | 0.07 | 0.20 | 0.50 | 1.90 | 4.07 |
| Break (J) | | | | | |
| Appearance | Very | Hazy | Hazy | Optically | Optically |
| of films | hazy | | | Clear | Clear |

*Distilled at 2 × 10⁻² mmHg and at 115° C.

It can also be determined by the contents of Table 1 that the PIB diol concentration affects the molecular weight and the mechanical properties of PIB-PUs in as much as Experiment 5 produced a PIB-PU having the best molecular weight and mechanical properties of any Experiment. It is also important to note that Experiment 5 was done under the exact same conditions as Experiment 4 (namely it also used freshly distilled MDI) and it produced better properties than Experiment 4. It should also be noted that during Experiment 5, that due to the concentration of the PIB polymer being greater than 21.4 wt. % in THF, the system became extremely viscous and stirring became impossible after about 20 minutes after the addition of the chain extender to the mixture. Past this point, stirring was able to be resumed from about 3 additional hours upon the addition of an additional 2 to 3 mL of THF.

Furthermore, by comparing the results of Experiment 4 with the results of Experiments 1-3, it can be seen what affect the use of freshly distilled MDI has on the properties of the PUB-PU's produced.

The molecular weights and the molecular weight distributions of the PIB-PU's documented in Table 1 were determined by gel permeation chromatography and structures were analyzed by ¹H NMR spectroscopy. Stress-Strain traces of the PIB-PU's documented in Table 1 were obtained by Instron, Model 5543, Universal Tester, controlled by Blue Hill software. Specifically, dumbbell shaped samples (25 mm long and 3.1 mm wide at the neck) were used, the extension rate was 100 mm/min, and the results shown were averages of 3 determinations. To study the fatigue performance of the PIB-PU's documented in Table 1, crack nucleation experiments were conducted using ISO 37-2 dog-bone shaped samples prepared from 30×70 nm sheets of solvent cast films. An Instron ElectroPuls E10000 was used to cycle samples under displacement control at 1 Hz frequency at 50% and 100% strain.

The mechanical properties of polyurethanes are strongly affected by the purity of the diisocyanate and other reagents. Isocyanates, because of their extremely high reactivity, are particularly prone to react with impurities such as moisture and, particularly after lengthy storage times, they begin to contain slow forming impurities, such as dimers, oligomers, and polyureas. Thus, the shelf life of the MDI prior to use affected the purity of the end-products. Impurities reduce the concentration of the isocyanate function, and obviate the precise stoichiometry needed to produce the highest quality end-product. Slowly forming isocyanate oligomers and polyureas are particularly onerous as they can enter the hard segments and disrupt their morphology, thus compromising not only the mechanical properties, but also the optical properties.

As briefly discussed above, inspection of the results of Experiments 1-4 in Table 1 shows that the use of freshly distilled MDI in place of as-received MDI more than doubled molecular weights (from 21 to 56 kDa), significantly increased stress and elongation (from 9 MPa to 22 MPa, and from 110% to 480%, respectively), and close to tripled the toughness (from 0.07 to 1.9 J). The shelf life of MDI is also an important purity issue as it was observed that after vacuum distillation of as-received MDI, a white insoluble reside remained in the distillation flask. Similarly, a white precipitate appeared after a few days of MDI was stored at −12° C., and the amount of the white precipitate only increased with time.

The impurities discussed above that were found in the MDI were identified by ¹H NMR spectroscopy and showed the spectrum of freshly distilled MDI and showed the methylene protons that formed at 3.90 ppm and the aromatic protons that formed at 6.99-7.14 ppm. The resonances of the aromatic protons ortho and meta to the urea group also appeared at 7.42-7.52 ppm. NMR spectra were also taken which clearly showed the polyurea impurities in the MDI that arise during extended storage, can be almost entirely eliminated by routine distillation.

As briefly discussed above, the results of the Experiments documented in Table 1, were also used to explore the effect of the PIB-diol concentration on key mechanical properties of PIB-PU's. Thus, the concentration of the PIB-diol was increased from 21.4 to 28.5 wt. % in THF in Experiment 5 as compared to Experiments 1-4. In Experiments 1-4, the polymerizing systems became increasingly viscous, but stirring remained satisfactory. In contract, with Experiment 5, the system became extremely viscous and stirring became impossible about 20 minutes after the addition of the butane diol chain extender. However, it was found that stirring could be resumed for up to about 3 additional hours by diluting the system with 2-3 mL of THF.

As shown by the data in Table 1, namely that of Experiments 4 and 5, key mechanical properties increased significantly by increasing the PIB-diol concentration from 21.4 to 28.5 wt. % in THF. Stress at break increased from about 22 to about 32 MPA, elongation increased from 480 to 630%, and toughness doubled. Films made at the higher PIB-diol concentration were also found to be colorless and optically clear. It is thought that higher synthesis solution concentration increased the rate and extent of the reaction, leading to higher PIB-PU molecular weights and superior mechanical properties.

GPC traces were prepared and compared for PIB-diols and a PIB-PU made in accordance with Experiment 5. An exceedingly high molecular weight of the PIB-PU made in accordance with Experiment 5 suggested essentially complete stoichiometric chain extension. High molecular weights produce significantly enhanced levels of entanglement and catenation and led to observed high elastic moduli.

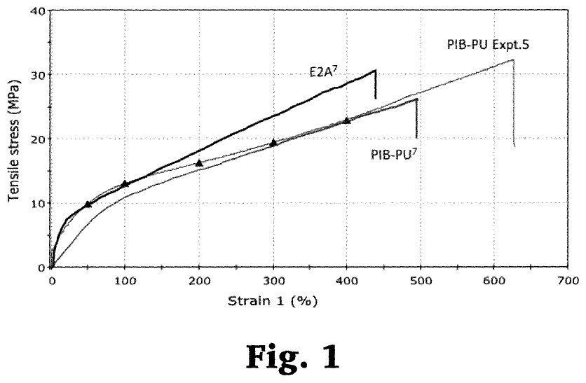

FIG. 1 shows stress vs. Strain traces of PIB-PU's obtained from Experiment 4 reported above, the highest quality PIB-PU reported to date (as disclosed in “Polyisobutylene-Based Polyurethanes: VII. Structure/Property Investigations for Medical Applications” by Toth et al. Journal of Polymer Science, Part A: Polymer Chemistry 2016, 54, 532-543), and a commercial silicon rubber-based polyurethane (Elast-Eon™). It is noted that a difference between the previously highest quality PIB-PU's reported, and the PIB-PU's of the present invention, is that the previously highest quality PIB-PU's reported did not utilize freshly distilled MDI in their creation and did not focus on the concentration of PIB polymer.

The stress/strain trace of the PIB-PU's start with a reversible elastic (Hookian) region up to about a 50% yield, followed by a high modulus deformation region, which suggests the strengthening of the hard segments by alignment and ordering until failure. High modulus and continuously increasing Young moduli are characteristic of highly elastic through materials. In respect to mechanical behavior, the PIB-PU of the present invention, although it contains only 30 wt. % hard segment, is superior as compared to the Elast-Eon™ polysiloxane-based PU having a 52 wt. % hard segment. In comparison to the highest quality PIB-PU reported to date, also having a 30 wt. % hard segment, due to its higher molecular weight and therefore higher extents of entanglements and/or catenation, the PIB-PU of the present invention exhibits superior strength, elongation, and toughness.

A key aspect of a polymer's properties for an application in a prosthetic, such as a prosthetic heart valve, is fatigue lifetime. Even more key is the fatigue lifetime over many cycles at low strain. Typical maximum strain experienced by a polymer in a heart valve leaflet is around 10% (with a maximum strain energy density of 0.05 MPa or less) and the lifetime must be 25 years or more (which equates to about 1 billion cycles). It is desirable to test the polymer at a similar frequency to that experienced in practice (about 1 Hz). As it is impractical to test fatigue to failure in at a realistic strain, there is a compromise required between the strain used for testing and the time taken to complete the experiment. Typically, strains of 50-100% are used.

Loading PIB-PU samples under cyclic fatigue leads to stress softening and therefore elongation, which is partially recovered upon unloading. Samples were tested until excessive elongation prevented further cycling at close to the desired strain, and the displacement was then re-set to return the sample to the nominal strain required (50% or 100%) and the test was continued. As a result, a range of strains was achieved over the course of the experiment as the sample crept and was then re-set. It was interestingly observed that this creep was at least largely reversible after completion of the experimental test. The results of this experiment are detailed below in Table 2.

TABLE 2
Based upon composition from Experiment 5

| Strain | | Elongation | Force | SED Range |
|---|---|---|---|---|
| (%) | Cycles | (%) | (N) | (MPa) |
| 50 | 910,633 | 11 | 6.5-8.5 | 1.15-1.18 |
| 100 | 590,185 | 91 | 12-14 | 2.02-3.51 |

The strain energy density (SED) determined by the integrated area under the stress-strain curve is given as a range for the reasons described above. It was notable that creep was much more pronounced at 100% strain than at 50% strain, which gives reason to believe that creep is unlikely to be an issue at the low strains required of a polymeric heart valve leaflet.

Fatigue life is typically expressed as the number of cycles to failure at a given strain. However, in this study, failure was unable to be achieved, even at relatively high levels of strain. At 50% strain, which is far beyond that experienced by a polymeric heart leaflet (max of about 10% strain) the approximate number of cycles without failure is about 1,000,000 cycles. However, because failure was not achieved at 50% strain within a reasonable timeframe, the nominal strain was increased to 100%, but again failure was not achieved, even after close to 600,000 cycles.

Higher strains were not attempted because the increasing creep with higher strains makes the experiments impractical. Based on the above data, one can deduce that at smaller strains (about 10%) many more cycles will occur before fatigue failure. This extremely high number of cycles without failure in the PIB-PU's of the present invention as compared, for example, to SIBS or SEBS which achieve a reported less than 100,000 cycles at SED around 1 MPa (according to Eugenia Biral, PhD Thesis, University of Cambridge, 2021) suggests that the PIB-PU's of the present invention can be expected to have excellent fatigue life

In sum, the use of purified MDI combined with the increased PIB-diol concentration led to the production of PIB-PU's with heretofore unseen levels of ultimate strength, elongation, and toughness. These key properties lead to new fatigue resistant PIB-PU's suitable for synthetic heart leaflets, that could not be made from earlier produced PIB-PU's.

In light of the foregoing, it should be appreciated that the present invention significantly advances the art by providing a heart valve that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A method of preparing a polyisobutylene-based polyurethane comprising the steps of:
a. providing a polyisobutylene (PIB) polymer;
b. freshly distilling a diisocyanate compound to create a freshly distilled diisocyanate;
c. providing a chain extender; and
d. combining the polyisobutylene polymer, the freshly distilled diisocyanate, and the chain extender by mixing to create the polyisobutylene-based polyurethane compound and wherein said polyisobutylene-based compound exhibits a higher number average molecular weight, a higher ultimate strength, a higher elongation, and a greater toughness than a polyisobutylene-based polyurethane made without a freshly distilled diisocyanate.

2. The method of claim 1, further comprising the steps of providing a catalyst and combining said catalyst with the polyisobutylene polymer, the freshly distilled diisocyanate, and the chain extender.

3. The method of claim 2, wherein the step of providing a catalyst includes selecting the catalyst from the group consisting of Dibutyltin dilaurate (DBTDL), stannous octoate, bismuth/zinc, zirconium and bismuth organics including bismuth neodecanoate, zinc neodecanoate, zinc carboxylate, and bismuth carboxylate, vanadium organics, and cobalt organics.

4. The method of claim 1, wherein the step of providing PIB polymer includes selecting a PIB-diol as the PIB polymer.

5. The method of claim 1, wherein the step of freshly distilling a diisocyanate includes selecting methylene diphenyl diisocyanate to be the freshly distilled diisocyanate.

6. The method of claim 1, wherein the step of providing a chain extender includes selecting butane diol as the chain extender.

7. The method of claim 1, wherein the step of combining produces a polyisobutylene-based polyurethane having a number average molecular weight of greater than 100,000 Da.

8. The method of claim 1, wherein the step of combining produces a polyisobutylene-based polyurethane having an ultimate strength of greater than 30 MPa.

9. The method of claim 1, wherein the step of combining produces a polyisobutylene-based polyurethane having an elongation of greater than 600%.

10. The method of claim 1, wherein the step of combining produces a polyisobutylene-based polyurethane having a toughness of greater than 4.00 J.

11. The method of claim 1, wherein the step of providing PIB polymer includes providing said PIB polymer in a solution of THF and wherein the concentration of said PIB polymer is at least 21.4 wt. % in THF.

12. The method of claim 11, wherein the step of providing PIB polymer includes providing said PIB polymer in a solution of THF at a concentration of greater than 21.4 wt. % in THF such that mixing during the combining step becomes impossible.

13. A polyisobutylene-based polyurethane comprising the reaction product of:
a. a polyisobutylene (PIB) polymer,
b. a freshly distilled diisocyanate compound, and
c. a chain extender,
wherein the polyisobutylene-based polyurethane exhibits a higher number average molecular weight, a higher ultimate strength, a higher elongation, and a greater toughness than a polyisobutylene-based polyurethane made without a freshly distilled diisocyanate.

14. The polyisobutylene-based polyurethane of claim 13, wherein the reaction product further includes a catalyst selected from the group consisting of Dibutyltin dilaurate (DBTDL), stannous octoate, bismuth/zinc, zirconium and bismuth organics including bismuth neodecanoate, zinc neodecanoate, zinc carboxylate, and bismuth carboxylate, vanadium organics, and cobalt organics.

15. The polyisobutylene-based polyurethane of claim 13, wherein the polyisobutylene polymer is a PIB-diol.

16. The polyisobutylene-based polyurethane of claim 13, wherein the freshly distilled diisocyanate compound is methylene diphenyl diisocyanate.

17. The polyisobutylene-based polyurethane of claim 13, wherein the polyisobutylene-based polyurethane has a number average molecular weight of greater than 100,000 Da, an ultimate strength of greater than 30 MPa, an elongation of greater than 600%, and a toughness of greater than 4.00 J.

18. A heart valve comprising the polyisobutylene-based polyurethane of claim 13.

19. A method for producing a heart valve, including the step of preparing a polyisobutylene-based polyurethane comprising the steps of:
providing a polyisobutylene (PIB) polymer;
freshly distilling a diisocyanate compound to create a freshly distilled diisocyanate;
providing a chain extender; and
combining the polyisobutylene polymer, the freshly distilled diisocyanate, and the chain extender by mixing to create the polyisobutylene-based polyurethane compound and wherein said polyisobutylene-based compound exhibits a higher number average molecular weight, a higher ultimate strength, a higher elongation, and a greater toughness than a polyisobutylene-based polyurethane made without a freshly distilled diisocyanate.

20. A method for producing a heart valve as set forth in claim 19, wherein the step of providing PIB polymer includes providing said PIB polymer in a solution of THF at a concentration of greater than 21.4 wt. % in THF such that mixing during the combining step becomes impossible
